(19) United States
(12) Patent Application Publication
(10) Pub. No.: US 2009/0270626 A1
(43) Pub. Date: Oct. 29, 2009

(54) STABLE BISTHIOL COMPOUNDS FOR RADIOIMAGING AND THERAPY

(75) Inventor: Peter C. Meltzer, Lexington, MA (US)

Correspondence Address: EDWARDS ANGELL PALMER & DODGE LLP, P.O. BOX 55874, BOSTON, MA, 02205, US

(73) Assignee: Organix, Inc., Woburn, MA (US)

(21) Appl. No.: 12/403,355

(22) Filed: Mar. 12, 2009

Related U.S. Application Data
(60) Provisional application No. 61/036,072, filed on Mar. 12, 2008.

(51) Int. Cl. C07D 471/08

(52) U.S. Cl. 546/112

(57) ABSTRACT

The invention provides compounds and methods for preparing radiopharmaceutical compounds useful as therapeutic and diagnostic agents.

### CROSS-REFERENCE TO RELATED APPLICATIONS

This application claims the benefit of U.S. Provisional Patent Application No. 61/036,072, filed Mar. 12, 2008, the contents of which are incorporated herein by reference.

### BACKGROUND OF THE INVENTION

Thiols are generally known to be susceptible to oxidative coupling to form disulfides. Compounds that contain two or more thiol moieties may have the capacity to form both inter- as well as intramolecular disulfides. Consequently, bisthiol compounds, including compounds containing certain metal chelating moieties, that contain at least two sulfhydryl (thiol) groups can readily undergo disulfide formation, thus rendering them ineffective in subsequent metal chelating reactions.

This instability of the bisthiols has been noted in the literature for thiol-containing metal chelating ligands including both MAMA and MAMA′ ligands. The chelating agent, MAMA′, is a monoamine monoamide bisthiol and has been synthesized as a thiol-protected entity incorporating protecting groups such as trityl groups to prevent disulfide formation. The protecting groups are then removed immediately prior to the introduction of technetium or rhenium, e.g., for compounds used as diagnostic imaging agents. Therefore, the incorporation of rhenium or technetium in metal chelating ligands such as monoaminemonoamide dithiols (MAMA or MAMA′) has generally been conducted in a one-pot procedure that involves deprotection of the bisthiol moieties to provide the bisthiols in situ, followed immediately by introduction of the metal (Re or ^{99m}Tc).

While such one-pot methods of deprotection and incorporation of the radiometal can be effective to produce the desired metal complexes, they can suffer from a number of disadvantages, such as inconsistent yields of metal incorporation; slow deprotection of the second thiol group; difficulty in monitoring the progress of the deprotection step; formation of complex mixtures that can require careful purification prior to use; and limited solubility in water or in buffer of the protected precursors.

### SUMMARY OF THE INVENTION

In one aspect, the present invention relates to a crystalline form of a bisthiol compound. In one embodiment, the bisthiol compound is a salt, such as a dihydrochloride salt.

In another aspect, the bisthiol compound is in any suitable solid form. In one embodiment the crystalline form is a polymorph, pseudopolymorph, or in an amorphous state.

In another aspect, the present invention relates to a method of making a solid (e.g. crystalline) form of certain bisthiol compounds.

In one aspect, the invention provides a solid form of a compound of Formula I or Formula II:

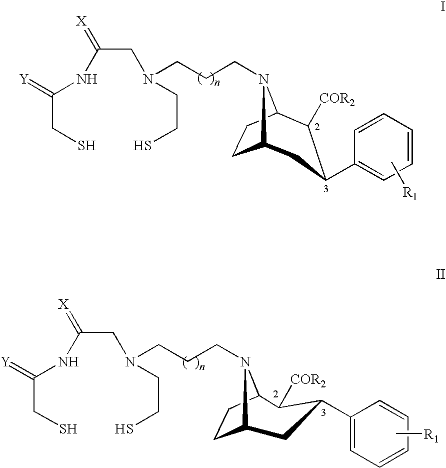

wherein:
the configuration at C₂ and C₃ are independently α or β;
n is 0-3;
X is H, H or O;
Y is H, H, or O;
R₁ is represents 3-R₃, 4-R₃, or 3,4-diR₃;
R₂ is —OR₄ or C₁-C₆alkyl;
R₃ is independently selected for each occurrence from the group consisting of halogen, hydroxyl, C₁-C₆alkyl; C₂-C₆alkenyl, or C₂-C₆alkynyl;
R₄ is C₁-C₆alkyl;
or a pharmaceutically acceptable salt thereof.

In certain embodiments, R₃ is chloro or fluoro.

In one embodiment, the bisthiol compound is N-[2-(3′-N′-Propyl-3″α-(4-fluorophenyl)tropane-2″β-(1-propanoyl)-[(2-mercapto-ethyl)-amino]-(2-mercapto-ethyl-acetamide, represented by the formula:

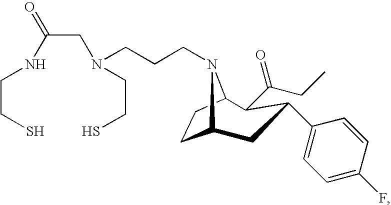

or a pharmaceutically acceptable salt thereof.

In another embodiment the bisthiol compound is N-[2-(3′-N′-Propyl-3″β-(4-fluorophenyl)tropane-2″β-(1-carboxylic acid methyl ester)-[(2-mercapto-ethyl)-amino]-(2-mercapto-ethyl-acetamide, represented by the formula:

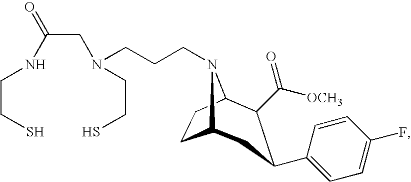

or a pharmaceutically acceptable salt thereof.

In further embodiments, the invention provides a compound of Formula I or II in a substantially isolated and/or purified form, e.g., in a solution, e.g., in a pharmaceutically acceptable carrier.

In a further embodiment, the invention provides a method for preparing a solid, isolated, and/or purified compound of the invention.

In a further embodiment, the invention provides a method for preparing a complex of a compound of Formula I or II together with a metal, e.g., a radionuclide.

In another aspect, the invention provides a kit for the preparation of a radiopharmaceutical, the kit comprising a solid, purified, and/or isolated form of a compound of Formula I or II in a container, together with instructions for complexing a radioisotope to the compound to prepare a radiopharmaceutical.

### DETAILED DESCRIPTION OF THE INVENTION

In one aspect, the present invention relates to a crystalline form of a bisthiol compound, e.g., a compound of Formula I or Formula II, or a pharmaceutically acceptable salt thereof. In one embodiment, the bisthiol compound is a salt, such as a dihydrochloride salt.

In another aspect, the bisthiol compound is in any suitable solid form. In one embodiment the crystalline form is a polymorph, pseudopolymorph, or in an amorphous state.

In another aspect, the present invention relates to a method of making a solid, isolated, and/or purified (e.g., crystalline) form of certain bisthiol compounds.

In one aspect, the invention provides a solid form of a compound of Formula I or Formula II:

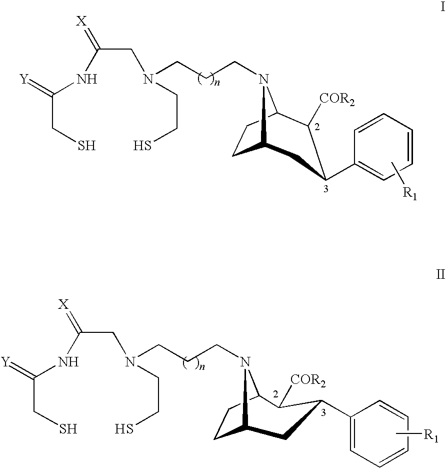

wherein:
the configuration at C₂ and C₃ are independently α or β;
n is 0-3;
X is H, H or O;
Y is H, H, or O;
R₁ is represents 3-R₃, 4-R₃, or 3,4-diR₃;
R₂ is —OR₄ or C₁-C₆alkyl;
R₃ is independently selected for each occurrence from the group consisting of halogen, hydroxyl, C₁-C₆alkyl; C₂-C₆alkenyl, or C₂-C₆alkynyl;
R₄ is C₁-C₆alkyl;
or a pharmaceutically acceptable salt thereof.

In certain embodiments, R₃ is chloro or fluoro. In certain embodiments, the solid form is a pharmaceutically acceptable salt. In certain embodiments, the pharmaceutically acceptable salt is a hydrochloride salt. In certain embodiments, the hydrochloride salt is a monohydrochloride or a dihydrochloride. In certain embodiments, the configuration at C₂ is β. In certain embodiments, the configuration at C₃ is α. In certain embodiments, the configuration at C₂ is β. In certain embodiments, the stereochemistry of the compound is either 1R or 1S. In certain embodiments, the stereochemistry of the compound is 1R. In certain embodiments, the stereochemistry of the compound is 1S.

In one embodiment, the bisthiol compound is represented by the formula:

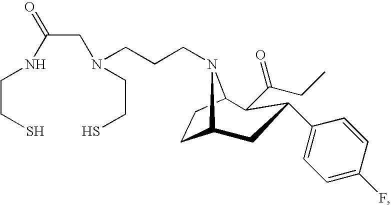

or a pharmaceutically acceptable salt thereof. In certain embodiments, the form of the compound is a pharmaceutically acceptable salt. In certain embodiments, the pharmaceutically acceptable salt is a hydrochloride salt. In certain embodiments, the hydrochloride salt is a monohydrochloride or a dihydrochloride. In certain embodiments, the stereochemistry of the compound is either 1R or 1S.

In further embodiments, the invention provides a compound of Formula I or II in a substantially isolated and/or purified form, e.g., in a solution, e.g., in a pharmaceutically acceptable carrier.

In another embodiment the bisthiol compound is represented by the formula:

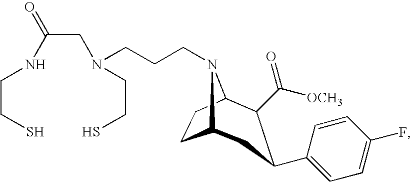

or a pharmaceutically acceptable salt thereof. In certain embodiments, the form of the compound is a pharmaceutically acceptable salt. In certain embodiments, the pharmaceutically acceptable salt is a hydrochloride salt. In certain embodiments, the hydrochloride salt is a monohydrochloride or a dihydrochloride. In certain embodiments, the stereochemistry of the compound is either 1R or 1S.

In a further embodiment, the invention provides a method for preparing a solid, isolated, and/or purified compound of the invention. In one embodiment, the method comprises contacting a thiol-protected (e.g., trityl-protected) precursor of the compound of Formula I or Formula II with a trialkylsilane in the presence of trifluoroacetic acid, such that a compound of Formula I or Formula II is prepared. In certain embodiments, the trialkylsilane is triethylsilane. In certain embodiments, the trityl-protected precursor of the compound of Formula I or Formula II is a compound of Formula Ia or Ia:

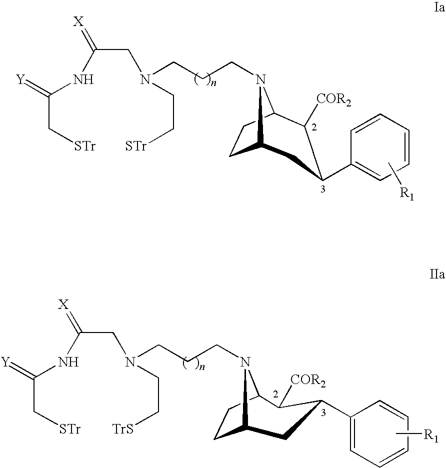

in which R₁, R₂, X, Y, n, and the configuration at C₂ and C₃ are as described for Formula I and II, and Tr is trityl (triphenylmethyl). In certain embodiments, the method includes the step of isolating or purifying the compound of Formula I or Formula II. In certain embodiments, the method includes the step of removing a solvent to isolate the compound of Formula I or Formula II.

The invention also provides a kit for the preparation of a radiopharmaceutical, the kit comprising a solid, purified, and/or isolated form of a compound of Formula I or II in a container, together with instructions for complexing a radioisotope to the compound to prepare a radiopharmaceutical. In certain embodiments, the container is a pyrogen-free, sterilized container, such as a vial.

It has now been found that compounds according to Formula I or II are stable in solid form or even in solution for extended periods. In certain embodiments, a solid, isolated and/or purified compound or composition of the invention is stable (i.e., is not substantially degraded and does not form significant amounts of a disulfide product) for at least 10 hours, 20 hours, 1 day, 2 days, 3 days, 4 days, 5 days, 6 days, or 7 days (or more). In certain embodiments, a solid, isolated and/or purified compound or composition of the invention is stable in the presence of air.

In a further embodiment, the invention provides a method for preparing a complex of a compound of Formula I or II together with a metal, e.g., a radionuclide.

Tropane derivative compounds useful for the preparation of compounds of Formula I and II can be prepared according to methods known in the art or as described herein. For example, precursor compounds having the two thiol groups of Formula I and II protected using a thiol protecting group (for example, a trityl group) can be prepared according to methods described in U.S. Pat. No. 7,105,678 and references cited therein; this patent is incorporated herein by reference in its entirety. Such protected precursor compounds can be deprotected to provide a compound according to Formula I or II using deprotection conditions, e.g., as known in the art (see, e.g., Greene and Wuts, eds. “Protective Groups in Organic Synthesis,” Third Edition, John Wiley & Sons, Inc., New York, 1999), or as described herein.

As used herein, the term “alkyl” refers to a straight-chained or branched hydrocarbon group containing 1 to 6 carbon atoms. Examples of alkyl groups include methyl, ethyl, n-propyl, isopropyl, tert-butyl, and n-pentyl. Alkyl groups may be optionally substituted with one or more substituents such halogen, hydroxyl, or alkoxy.

The term “alkenyl” refers to an unsaturated hydrocarbon chain that may be a straight chain or branched chain, containing 2 to 6 carbon atoms and at least one carbon-carbon double bond. Alkenyl groups may be optionally substituted with one or more substituents as for alkyl groups.

The term “alkynyl” refers to an unsaturated hydrocarbon chain that may be a straight chain or branched chain, containing the 2 to 6 carbon atoms and at least one carbon-carbon triple bond. Alkynyl groups may be optionally substituted with one or more substituents as for alkyl groups.

As used herein, the term “halogen” refers to Cl, F, Br, or I.

As used herein, the term “trityl” refers to a triphenylmethyl group.

Some of the compounds of this invention have one or more double bonds, or one or more asymmetric centers. Such compounds can occur as racemates, racemic mixtures, single enantiomers, individual diastereomers, diastereomeric mixtures, and cis- or trans- or E- or Z-double isomeric forms. All such isomeric forms of these compounds are expressly included in the present invention.

As used herein, an isolated and/or purified form of a compound of Formula I or II refers to a compound which has been substantially isolated, i.e., separated from impurities, or substantially purified, e.g., by chromatography, crystallization, or other methods known in the art. In certain embodiments, an isolated or purified compound or composition of the invention is substantially free of impurities such as, e.g., residues of protecting groups resulting from deprotection of thiol protected precursor compound, including, e.g., triphenylmethanol. The purity of a purified compound or composition of the invention can be, e.g., 70%, 80%, 90%, 95% or greater.

The compounds of the invention can be prepared either as free bases or as a salt thereof. Preferred salts are pharmaceutically acceptable salts, i.e., salts suitable for use in a pharmaceutical formulation. Such salts should have low toxicity and should not unduly interfere with the intended purpose of the compound or formulation. Representative salts include, but are not limited to, hydrochloride, hydrobromide, sulfate, bisulfate, acetate, valerate, oleate, palmatate, stearate, laurate, borate, benzoate, lactate, phosphate, tosylate, mesylate, citrate, maleate, fumarate, succinate, tartrate, glucoheptonate, lactobionate, naphthalene-1,5-disulfonate, lauryl sulfate salts and the like.

In one embodiment, the composition is a solid composition. In another embodiment the solid compound or composition is in a crystalline form. In another embodiment the solid composition is in a “solvent-free form,” i.e., a form substantially free of solvents. In another embodiment the solid composition is anhydrous. In another embodiment, the solid composition is in a pyrogen-free, sterilized container or vial. The container or vial can be unit dose or multi-dose.

Polymorphism and pseudopolymorphism are known in the pharmaceutical sciences. For a general review of polymorphs and the pharmaceutical applications of polymorphs see G. M. Wall, Pharm Manuf. 3, 33 (1986); J. K. Haleblian and W. McCrone, J. Pharm. Sci., 58, 911 (1969); and J. K. Haleblian, J. Pharm. Sci., 64, 1269 (1975), all of which are incorporated herein by reference. Many organic compounds can crystallize in more than one type of molecular packing with more than one type of internal crystal lattice. The respective resulting crystal structures can have, for example, different unit cells. This phenomenon, identical chemical structure but different internal structure, is referred to as polymorphism and the species having different molecular structures are referred to as polymorphs.

Many pharmacologically active organic compounds can also crystallize such that second, foreign molecules, for example solvent molecules, are incorporated into the crystal structure of the principal compound. This phenomenon is referred to as pseudopolymorphism and the resulting structures as pseudopolymorphs. When the second molecule is a solvent molecule, the pseudopolymorphs can be referred to as solvates.

The compounds or compositions of the invention can be used to prepare metal complexes, which can be used as therapeutic or diagnostic agents. See, e.g., U.S. Pat. No. 7,105,678 and references cited therein; this patent is incorporated herein by reference in its entirety. In general, a compound or composition of Formula I or II is contacted with a metal (e.g., a radio-nuclide such as Tc^{99m}) or a metal chelate under conditions that allow the metal to become complexed to the dithiol chelating moiety. Such complexation reactions generally occur in solution, often in the presence of a reducing agent. For example, to form a Tc^{99m} complex, a pertechnetate salt can be used, together with a compound or composition of the invention, e.g., in the presence of a reducing agent such as tin(II) chloride. In another procedure, a transchelation agent such as glucoheptonate (gluceptate) (e.g., from a DRAXImage kit, DRAXIMAGE, Quebec, Canada) as the metal complex (e.g., Tc^{99m} gluceptate) is used together with a compound or composition of Formula I or II to form a metal complex. In certain embodiments, the metal is rhenium or technetium; in certain embodiments, the metal is Tc^{99m}.

Without further elaboration, it is believed that the above description has adequately enabled the present invention. The following specific embodiments are, therefore, to be construed as merely illustrative, and not limitative of the remainder of the disclosure in any way whatsoever.

All of the references and publications cited herein are hereby incorporated by reference in their entirety.

### EXAMPLES

It has now been found that the bisthiol free base of O-5648 was stable up to 100 hours at room temperature in methanol exposed to air. The bisthiol dihydrochloride salt O-5648 was stable in MeOH up to 150 hours at room temperature. The solid bisthiol dihydrochloride salt exposed to air was surprisingly stable for >170 hours at room temperature. The solid bisthiol dihydrochloride salt O-5648 can be stored for long periods as a stable crystalline solid. It can be readily incorporated in prepared kits for clinical use. Furthermore, the incorporation of rhenium and technetium into the solid bisthiol precursors can be easily accomplished in high chemical and radiochemical yields. Consequently the bisthiol dihydrochloride precursors are superior to the free base ligands for incorporation of ^{99m}Tc in the clinical setting.

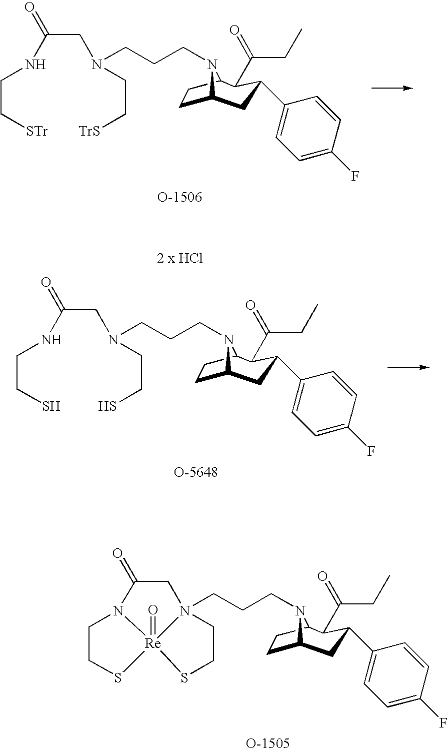

### Experimental Procedures

The bistrityl (protected) tropane derivatives described herein were synthesized as described by Meltzer et al. (Meltzer et al., J. Med. Chem., 1997, 40, 1835-1844; Meltzer et al., J. Med. Chem. 2003, 46, 3483-3496), or U.S. Pat. No. 7,105,678, each of which is incorporated herein by reference.

### Example 1

### Deprotection of bistrityl precursors O-1506 and O-912

### N-[2-(3′-N′-Propyl-(1″R)-3″α-(4-fluorophenyl)tropane-2″β-(1-propanoyl)-[(2-mercapto-ethyl)-amino]-(2-mercapto-ethyl-acetamide, dihydrochloride salt, O-5648

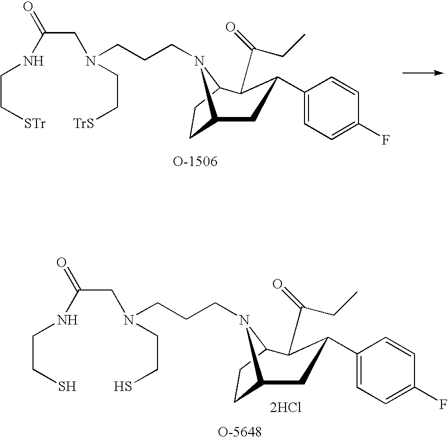

Trifluoroacetic acid (TFA, 1.33 mL, 17.3 mmol) was added drop wise at 0° C., under N₂ atmosphere to a solution of O-1506 (850 mg, 0.868 mmol) in anhydrous dichloromethane (62 mL) to obtain a yellow solution. To this reaction mixture, Et₃SiH (3.4 mL, 21.3 mmol) was added dropwise to obtain a pale yellow solution which was stirred at 0° C. for 30 min and at room temperature (ca. 22° C.) overnight. The reaction mixture was washed successively with 10% aq. Na₂CO₃ and saturated brine, dried using anhydrous Na₂SO₄ and evaporated. The crude product was purified by flash chromatography (eluent: ethyl acetate to EtOAc/MeOH (98:2) to EtOAc/MeOH/NH₄OH (97:3:1)) to afford 310 mg of product. The product was dissolved in CH₂Cl₂ and treated with 2M HCl in Et₂O (1.8 mL). Solvent was evaporated and the residue was triturated with ether and filtered. The compound isolated was transferred to a flask with methanol. The solvent was evaporated to leave a residue, which was dissolved in water and lyophilized to obtain O-5648 (340 mg, 69%) as a pale yellow solid. R_{f}=0.43 (EtOAc 92%, MeOH 4%, Conc. NH₃ 4%). MS [M+H]⁺ 496.1. ¹H NMR (300 MHz, D₂O) δ 7.41-7.46 (m, 2H), 7.11-7.17 (m, 2H), 4.14-4.19 (m, 3H), 3.99-4.03 (m, 1H), 3.33-3.64 (m, 7H), 3.09-3.20 (m, 3H), 2.88-2.94 (m, 2H), 2.68 (t, 2H, J=6.6 Hz), 1.97-2.62 (m, 10H), 0.84 (t, 3H, J=7.2 Hz). Anal. (C₂₅H₃₈FN₃O₂S₂.2.2HCl 1.8H₂O) C, H, N.

### Example 2

### N-[2-(3′-N′-Propyl-(1″R)-3″β-(4-fluorophenyl)tropane-2″β-(1-carboxylic acid methyl ester)-[(2-mercapto-ethyl)-amino]-(2-mercapto-ethyl-acetamide, dihydrochloride salt, O-6117

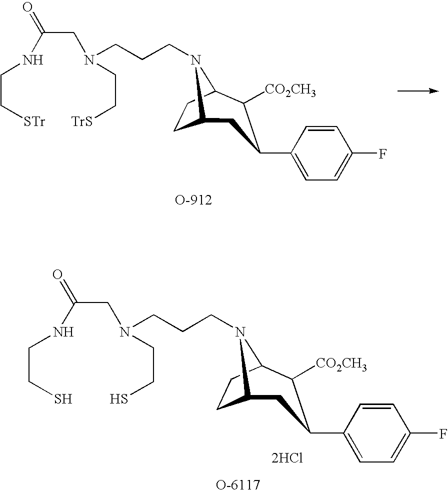

TFA (1.33 mL, 17.3 mmol) was added drop wise at 0° C., under N₂ atmosphere to a solution of O-912 (850 mg, 0.87 mmol) in anhydrous dichloromethane (62 mL) to obtain a yellow solution. To this reaction mixture, Et₃SiH (0.20 mL, 1.28 mmol) was added dropwise to obtain a pale yellow solution which was stirred at 0° C. for 30 min and at room temperature (ca. 22° C.) overnight. The reaction mixture was combined with an earlier batch of O-6117 (50 mg, 0.051 mmol) for purification. The combined reaction mixture was washed successively with 10% aq. Na₂CO₃ and saturated brine and evaporated. The crude product was purified by flash chromatography (eluent:ethyl acetate to EtOAc/MeOH/NH₄OH (96:4:2)) to afford 442 mg of product. The product was dissolved in CH₂Cl₂ and treated with 2M HCl in Et₂O (1.2 mL) and stirred at room temperature for 1 h. Evaporation of the solvent followed by drying in vacuum afforded O-6117 (513 mg, 98%) as a white foam. R_{f}=0.51 (EtOAc 92%, MeOH 4%, Conc. NH₃ 4%). MS [M+H]⁺ 498.1. ¹H NMR (300 MHz, D₂O) δ 7.21-7.26 (m, 2H), 7.08-7.14 (m, 2H), 4.33-4.35 (m, 1H), 4.16 (bs, 1H), 4.06 (s, 2H), 3.65-3.73 (m, 1H), 3.17-3.46 (m, 12H), 2.84-2.89 (m, 2H), 2.67 (t, 2H, J=6.3 Hz), 2.59-2.63 (m, 1H), 2.19-2.38 (m, 6H), 2.02-2.11 (m, 1H). Anal. (C₂₄H₃₆FN₃O₃S₂.2HCl.2.6H₂O) C, H, N.

### Example 3

### Stability of the Deprotected Bisthiol: O-5648

Stability of compound O-5648 (see Example 1, above) was measured by HPLC with UV and mass detection. The bisthiol free base was stable up to 100 hours at room temperature in methanol exposed to air.

The bisthiol dihydrochloride salt was stable in MeOH up to 150 hours at room temperature.

The solid bisthiol dihydrochloride salt exposed to air was stable for >170 hours at room temperature.

### Example 4

### Insertion of Rhenium into O-5648 Using Gluceptate Kits to Obtain O-1505

Insertion of rhenium was conducted on a scale commensurate with that planned for clinical insertion of technetium. Commercial Gluceptate kits (Draximage) contained approximately 0.7 mg (3.1 μmol) of SnCl₂.2H₂O, NaReO₄ (10.0 mg, 36.6 μmol) was dissolved in 5 mL of H₂O to provide a stock solution of 2.0 mg/mL. The experiment was performed twice using a 1:1:1 ratio and a 1:1:5 ratio of NaReO₄:SnCl₂.2H₂O: O-5648 respectively.

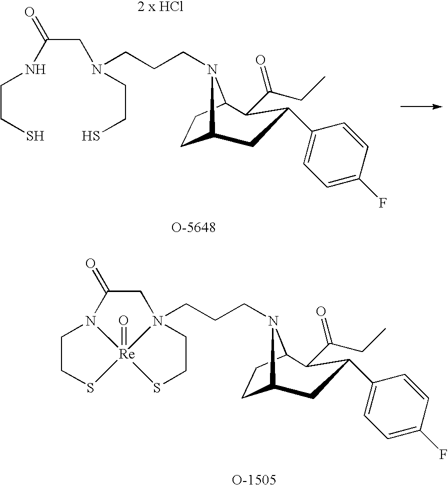

Stock NaReO₄ solution (0.43 mL, 0.86 mg, 3.15 μmol) was added to each of two of the gluceptate kits. O-5648 (1.8 mg, 3.1 μmol (1 eq) and 8.8 mg, 15.5 μmol (5 eq)) dissolved in water (0.57 mL−Total Vol=1.00 mL) were added to each of the two kits followed by 2 drops of concentrated HCl. The kits were heated on an oil bath at 90-95° C. for 2 h. On cooling, MeOH (1.00 mL) was added to each of the kits. An aliquot of the colorless supernatant liquid was extracted from the kits by syringe and filtered through a 0.45 μm filter for analysis. Yields were quantified by HPLC analysis (UV/MS). O-1505 (1 eq of ligand) Yield: 1.18 mg (50%); O-1505 (5 eq of ligand) Yield: 1.64 mg (69%).

### Example 5

### Insertion of Technetium into O-5648

Tc^{99m} gluceptate was prepared by injecting Tc^{99m} pertechnetate (124.4 mCi or 183.5 mCi) in 2×1.0 ml 0.9% saline solution into a vial of the DRAX Image gluceptate kit. O-5648 was prepared as an aqueous solution containing 0.57 mg/ml O-5648, 2.35 mg/ml sodium acetate trihydrate, 2.5 mg/ml sodium glucoheptonate, approximately 62 μm/ml tin(II) chloride dihydrate, pH 5.18.

One ml of the O-5648 solution was mixed with sodium Tc99m gluceptate solution in approximately 0.8 ml volume in a vial. After incubation at either 5° or 25° C., the mixture was analyzed by radioHPLC to determine the radiochemical purity of the metal complex. It was found that the radiochemical purity was about 83% after 7 hours at 5° and about 93% after about 2 hour 13 minutes at 25° C.

Insertion of Technetium into O-6117

O-6117 was prepared as an aqueous solution containing 0.50 mg/ml O-6117, 2.32 mg/ml sodium acetate trihydrate, 2.5 mg/ml sodium glucoheptonate, 0.51 mg/ml sodium ethylenediaminetetraacetate (EDTA), and approximately 72 μm/ml tin(II) chloride dihydrate, pH 5.32.

One ml of the O-6117 solution was mixed with sodium Tc^{99m} gluceptate solution in approximately 1.1 ml volume in a vial. After incubation at 25° C., the mixture was analyzed by radioHPLC to determine the radiochemical purity of the metal complex. It was found that the radiochemical purity was about 77% after about 8 hours 40 minutes at 25° C.

### Other Embodiments

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

What is claimed is:

1. A solid form of a compound of Formula I or Formula II: 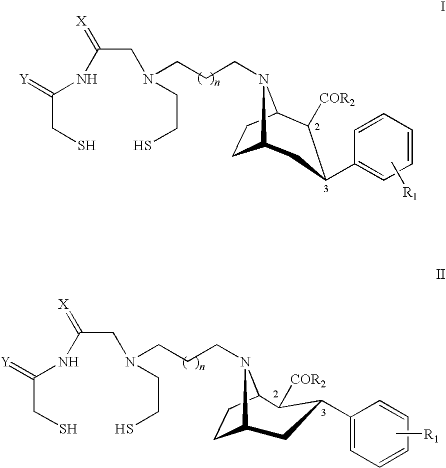
wherein:
the configuration at C₂ and C₃ are independently α or β;
n is 0-3;
X is H, H or O;
Y is H, H, or 0;
R₁ is represents 3-R₃, 4-R₃, or 3,4-diR₃;
R₂ is —OR₄ or C₁-C₆alkyl;
R₃ is halogen, hydroxyl, C₁-C₆alkyl; C₂-C₆alkenyl, or C₂-C₆alkynyl;
R₄ is C₁-C₆alkyl;
or a pharmaceutically acceptable salt thereof.

2. The solid form of claim 1, wherein the solid form is a pharmaceutically acceptable salt.

3. The solid form of claim 2, wherein the pharmaceutically acceptable salt is a hydrochloride salt.

4. The solid form of claim 3, wherein the hydrochloride salt is a monohydrochloride or a dihydrochloride.

5. The solid form of claim 1, wherein the stereochemistry of the compound is either 1R or 1S.

6. A method for preparing a compound of Formula I or Formula II: 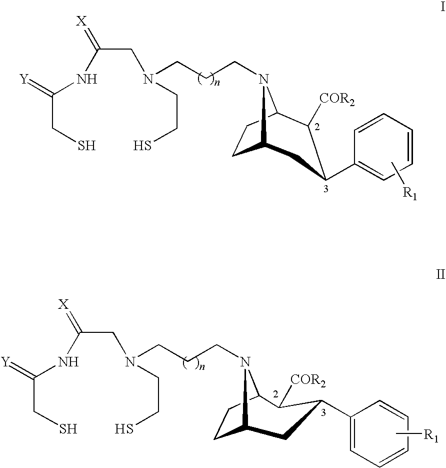
wherein:
the configuration at C₂ and C₃ are independently α or β;
n is 0-3;
X is H, H or O;
Y is H, H, or O;
R₁ is represents 3-R₃, 4-R₃, or 3,4-diR₃;
R₂ is —OR₄ or C₁-C₆alkyl;
R₃ is independently selected for each occurrence from the group consisting of halogen, hydroxyl, C₁-C₆alkyl; C₂-C₆alkenyl, or C₂-C₆alkynyl;
R₄ is C₁-C₆alkyl;
or a pharmaceutically acceptable salt thereof;
the method comprising contacting a trityl-protected precursor of the compound of Formula I or Formula II with a trialkylsilane in the presence of trifluoroacetic acid.

7. The method of claim 6, wherein the trialkylsilane is triethylsilane.

8. A kit for the preparation of a radiopharmaceutical, the kit comprising the solid form of claim 1 in a container, together with instructions for complexing a radioisotope to the compound of claim 1 to prepare a radiopharmaceutical.

9. An isolated compound represented by the formula: 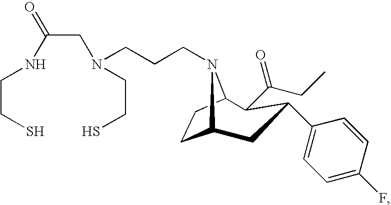
or a pharmaceutically acceptable salt thereof.

10. The isolated compound of claim 9, wherein the isolated compound is a solid form of the compound.

11. The isolated compound of claim 9 or 10, wherein the pharmaceutically acceptable salt is a hydrochloride salt.

12. The isolated compound of claim 11, wherein the hydrochloride salt is a monohydrochloride or a dihydrochloride.

13. The isolated compound of claim 12, wherein the hydrochloride salt is a dihydrochloride.

14. An isolated compound represented by the formula: 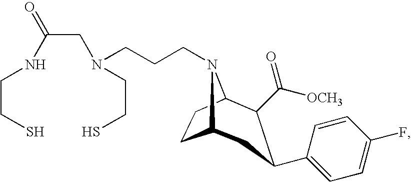
or a pharmaceutically acceptable salt thereof

15. The isolated compound of claim 14, wherein the isolated compound is a solid form of the compound.

16. The isolated compound of claim 14 or 15, wherein the pharmaceutically acceptable salt is a hydrochloride salt.

17. The isolated compound of claim 16, wherein the hydrochloride salt is a monohydrochloride or a dihydrochloride.

18. The isolated compound of claim 17, wherein the hydrochloride salt is a dihydrochloride.
